(19) United States
(12) Patent Application Publication
(10) Pub. No.: US 2011/0274636 A1
(43) Pub. Date: Nov. 10, 2011

(54) COMPOSITIONS CONTAINING ACRYLIC THICKENER AND HIGH VISCOSITY NON-SILICONE OIL

(75) Inventors: Gisela M. PERRUNA, Rahway, NJ (US); Dhaval PATEL, Edison, NJ (US); Hy Si BUI, Piscataway, NJ (US); Sara MUENZ, East Brunswick, NJ (US)

(73) Assignee: L'Oréal S.A., Paris (FR)

(21) Appl. No.: 13/101,303

(22) Filed: May 5, 2011

Related U.S. Application Data
(60) Provisional application No. 61/331,494, filed on May 5, 2010.
(60) Provisional application No. 61/331,543, filed on May 5, 2010.

(51) Int. Cl. A61K 8/81; A61Q 1/02; A61Q 1/04; A61Q 1/00

(52) U.S. Cl. 424/63; 514/772.6

(57) ABSTRACT

The invention relates to a composition, especially a cosmetic composition, comprising at least one acrylic thickener and at least one high viscosity non-silicone oil, as well as to methods of using such compositions.

### CROSS-REFERENCE TO RELATED APPLICATIONS

This application claims the benefit of priority under 35 U.S.C. §119(e) from U.S. Provisional Application Ser. Nos. 61/331,494 and 61/331,543, both filed May 5, 2010, the entire contents of which are hereby incorporated by reference.

### FIELD OF THE INVENTION

The present invention relates to compositions comprising at least one acrylic thickener and at least one high viscosity non-silicone oil. Among other improved or beneficial properties, compositions containing both an acrylic thickener and high viscosity oil have surprisingly good texture, spreadability, pick up and/or feel properties, as well as good skin adherence properties.

### DISCUSSION OF THE BACKGROUND

Many cosmetic compositions, including pigmented cosmetics such as foundations, concealers, lipsticks, and mascaras, and other cosmetic and sunscreen compositions, have been formulated in an attempt to posses good feel and texture upon application. Unfortunately, many of these compositions can be difficult to apply and do not possess a soft texture or smooth feel upon application. Moreover, such compositions oftentimes have a tendency to feel tacky, yielding poor application and spreadability characteristics.

Silicone elastomers have been added to cosmetic compositions to improve the feel of the compositions. However, the use of silicone elastomers can be problematic, given their expense and that they can be difficult to formulate owing to their chemical make up—for example, compositions including silicone elastomers can be unstable, particularly if uncomplimentary compounds are added to an elastomer-containing composition.

Thus, there remains a need for improved cosmetic compositions having improved cosmetic properties, particularly good feel and texture characteristics upon application, without relying solely (if at all) upon silicone elastomers.

Accordingly, one aspect of the present invention is a care and/or makeup and/or treatment composition for keratinous material which has good cosmetic properties such as, for example, good feel and/or texture properties upon application.

### SUMMARY OF THE INVENTION

The present invention relates to compositions comprising at least one acrylic thickener and at least one high viscosity non-silicone oil. Preferably, the compositions are free of silicone oil.

The present invention also relates to colored compositions comprising at least one coloring agent, at least one acrylic thickener, and at least one high viscosity non-silicone oil. Such colored compositions can be, for example, cosmetic compositions such as lip compositions (for example, lipstick or liquid lip colors), mascaras, eyeshadows or foundations. Preferably, the compositions are free of silicone oil.

The present invention further relates to compositions comprising at least one acrylic thickener, at least one high viscosity non-silicone oil, and water. Preferably, such compositions contain a significant amount of water (that is, at least 40%) and are in the form of an emulsion. Such water-containing compositions preferably further contain at least one coloring agent and are preferably foundations, lip compositions, blushes, eyeshadows, nail polishes or mascaras. Preferably, the compositions are free of silicone oil.

The present invention also relates to methods of treating, caring for and/or making up keratinous material (for example, skin, eyes, eyelashes or lips) by applying compositions of the present invention to the keratinous material in an amount sufficient to treat, care for and/or make up the keratinous material.

The present invention also relates to methods of enhancing the appearance of keratinous material (for example, skin, eyes, eyelashes, or lips) by applying compositions of the present invention to the keratinous material in an amount sufficient to enhance the appearance of the keratinous material.

The present invention further relates to compositions having improved cosmetic properties such as, for example, increased anti-smudging properties, increased long wear properties, and/or better texture or feel upon application.

The present invention also relates to methods of improving the feel or texture properties of a composition upon application to a keratin material comprising adding to a composition (for example, a foundation) at least one acrylic thickener and at least one high viscosity non-silicone oil.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

The composition of the present invention may be in any form, either liquid or non-liquid (semi-solid, soft solid, solid, etc.). For example, it may be a paste, a solid, a gel, or a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel. The composition of the invention may, for example, comprise an external or continuous fatty phase. The composition can also be a molded composition or cast as a stick or a dish.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf.

This hardness is measured in one of two ways. A first test for hardness is according to a method of penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The hardness of the composition of the present invention may be such that the compositions are self-supporting and can easily disintegrate to form a satisfactory deposit on keratin materials. In addition, this hardness may impart good impact strength to the inventive compositions, which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within preferred embodiments of the invention.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 25° C., 37° C., 45° C. and under freeze-thaw conditions. A composition is considered to lack stability if in any of these tests an abnormality that impedes functioning of the composition is observed. The skilled artisan will readily recognize an abnormality that impedes functioning of a composition based on the intended application.

The cosmetic compositions and methods of the present invention can comprise, consist of, or consist essentially of the essential elements and limitations of the invention described herein, as well as any additional or optional ingredients, components, or limitations described herein or otherwise useful in personal care.

Acrylic Thickener

According to the present invention, compositions comprising at least one acrylic thickener are provided. “Acrylic thickener” as used herein refers to polymers based upon one or more (meth)acrylic acid (and corresponding (meth)acrylate) monomers or similar monomers.

According to preferred embodiments, the acrylic thickener is an anionic acrylic polymer comprising at least one monomer performing a weak acid function such as, for example, acrylic acid, methacrylic acid, itaconic acid, crotonic acid, maleic acid and/or fumaric acid.

According to preferred embodiments, the acrylic thickener is an anionic acrylic polymer further comprising at least one monomer performing a strong acid function such as, for example, monomers having a function of the sulfonic acid type or phosphonic acid type, such as 2-acrylamido-2-methylpropane sulfonic acid (AMPS).

According to preferred embodiments, the anionic acrylic polymer may be crosslinked (or branched). Suitable examples of acceptable crosslinking agents include, but are not limited to, methylene bisacrylamide (MBA), ethylene glycol diacrylate, polyethylene glycol dimethacrylate, diacrylamide, cyanomethacrylate, vinyloxyethacrylate or methacrylate, formaldehyde, glyoxal, and compositions of the glycidylether type such as ethyleneglycol diglycidylether, or epoxydes.

Particularly preferred acrylic thickeners are disclosed in U.S. patent application publication nos. 2004/0028637 and 2008/0196174, the entire contents of both of which are incorporated herein by reference. Particularly preferred acrylic thickeners are sodium acrylate/sodium acryloyldimethyl taurate copolymers which. A particularly preferred acrylic thickener is that sold under the INCI name Sodium Acrylate/Sodium Acryloyldimethyl Taurate Copolymer & Hydrogenated Polydecene & Sorbitan Laurate & Trideceth-6 which is marketed by Arch Personal Care Products, South Plainfield, N.J., USA under the tradename ViscUp®EZ.

Preferably, the acrylic thickener(s) represent from about 0.1% to about 10% of the total weight of the composition, more preferably from about 0.2% to about 5% of the total weight of the composition, and most preferably from about 0.5% to about 2.5%, including all ranges and subranges therebetween.

### High Viscosity Non-Silicone Oil

According to the present invention, compositions comprising at least one high viscosity non-silicone oil are provided. Preferably, the at least one high viscosity non-silicone oil is a high viscosity ester oil, a high viscosity hydrocarbon oil, or a mixture of the two.

High Viscosity Ester Oil

In accordance with the present invention, “oil” means any non-aqueous medium which is liquid at ambient temperature (25° C.) and atmospheric pressure (760 mm Hg). By “high viscosity ester oil,” it is meant an oil having a viscosity of at least 100 cSt at 25° C. measured according standard ASTM D-445. According to preferred embodiments of the present invention, the high viscosity oil has a viscosity greater than 100 cSt, preferably greater than 150 cSt, preferably greater than 200 cSt, preferably greater than 250 cSt, and more preferably greater than 300 cSt, up to about 100,000 cSt, including all ranges and subranges therebetween.

Suitable examples of ester oils include, but are not limited to, esters such as linear fatty acid esters having a total carbon number ranging from 30 to 70, such as pentaerythrityl tetrapelargonate (MM=697.05 g/mol); hydroxy esters, such as diisostearyl malate (MM=639 g/mol); aromatic esters such as tridecyl trimellitate (MM=757.19 g/mol); esters of C24-C28 branched fatty acids or fatty alcohols, such as those described in EP-A-0 955 039, for example triisocetyl citrate (MM=856 g/mol), pentaerythrityl tetraisononanoate (MM=697.05 g/mol), glyceryl triisostearate (MM=891.51 g/mol), glyceryl 2-tridecyltetradecanoate (MM=1 143.98 g/mol), pentaerythrityl tetraisostearate (MM=1 202.02 g/mol), poly-2-glyceryl tetraisostearate (MM=1 232.04 g/mol) and pentaerythrityl 2-tetradecyltetradecanoate (MM=1 538.66 g/mol); and mixtures thereof. Suitable ester oils can also be described according to formula R₁COOR₂ in which R₁ represents a linear or branched higher fatty acid residue containing from 1 to 40 carbon atoms, including from 7 to 19 carbon atoms, and R₂ represents a branched hydrocarbon-based chain containing from 1 to 40 carbon atoms, including from 3 to 20 carbon atoms, with R₁+R₂≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters. A particularly preferred ester is diisostearyl malate.

According to preferred embodiments, the at least one high viscosity ester oil is present in the compositions of the present invention in an amount ranging from about 1% to about 25% by weight, more preferably from about 2% to about 20% by weight, and most preferably from about 7% to about 15% by weight, based on the total weight of the composition, including all ranges and subranges within these ranges.

According to preferred embodiments, the ratio by weight between the acrylic thickener and the high viscosity ester oil ranges from about 0.01 to 1, preferably from about 0.02 to 0.5, and more preferably from about 0.03 to about 0.3, including all ranges and subranges therebetween.

High Viscosity Hydrocarbon Oil

In accordance with the present invention, “oil” means any non-aqueous medium which is liquid at ambient temperature (25° C.) and atmospheric pressure (760 mm Hg). By “high viscosity hydrocarbon oil,” it is meant an oil having a viscosity of at least 100 cSt at 25° C. measured according standard ASTM D-445. According to preferred embodiments of the present invention, the high viscosity oil has a viscosity greater than 100 cSt, preferably greater than 150 cSt, preferably greater than 200 cSt, preferably greater than 250 cSt, and more preferably greater than 300 cSt, up to about 100,000 cSt, including all ranges and subranges therebetween

Suitable examples of hydrocarbon oils include, but are not limited to, non-volatile hydrocarbon fluids having a molecular mass of more than 500 g/mol, for example more than 600 g/mol, and for example more than 650 g/mol. By “hydrocarbon” compound, it is meant a compound comprising principally atoms of carbon and hydrogen and optionally one or more functional groups chosen from hydroxyl, ester, ether and carboxyl functions. These compounds are, according to one aspect, devoid of —Si—O— groups. Suitable examples of hydrocarbon fluids include, but are not limited to polybutylenes, such as Indopol H-100 (of molar mass or MM=965 g/mol), Indopol H-300 (MM=1340 g/mol), and Indopol H-1500 (MM=2160 g/mol), which are sold or manufactured by Amoco; hydrogenated polyisobutylenes, such as Panalane H-300 E, sold or manufactured by Amoco (M=1340 g/mol), Viseal 20000 sold or manufactured by Synteal (MM=6000 g/mol), and Rewopal PIB 1000, sold or manufactured by Witco (MM=1000 g/mol); polydecenes and hydrogenated polydecenes, such as Puresyn 10 (MM=723 g/mol) and Puresyn 150 (MM=9200 g/mol) sold or manufactured by Mobil Chemicals.

According to preferred embodiments, the at least one high viscosity hydrocarbon oil is present in the compositions of the present invention in an amount ranging from about 1% to about 25% by weight, more preferably from about 2% to about 20% by weight, and most preferably from about 7% to about 15% by weight, based on the total weight of the composition, including all ranges and subranges within these ranges.

According to preferred embodiments, the ratio by weight between the acrylic thickener and the high viscosity hydrocarbon oil ranges from about 0.01 to 1, preferably from about 0.02 to 0.5, and more preferably from about 0.03 to about 0.3, including all ranges and subranges therebetween.

Water

According to preferred embodiments of the present invention, the compositions of the present invention further comprise water. In such embodiments, embodiment water is preferably present in an amount ranging from about 30 to about 90%, preferably from about 40 to about 85%, and more preferably from about 50 to about 80% relative to the total weight of the composition, including all ranges and subranges therebetween. Preferably, such water-containing cosmetic compositions are lip compositions (for example, lipstick or liquid lip colors), foundations or mascaras, and are emulsions or dispersions.

According to particularly preferred embodiments, the compositions of the present invention are in the form of an emulsion. Suitable emulsion forms include but are not limited to oil-in-water, water-in-oil, oil-in-water-in-oil, water-in-oil-in-water and nanoemulsions (emulsions whose oil globules are of very fine particle size, that is to say that they have a number-average size of less than about 100 nanometers (nm)). Emulsions contain at least one oil phase and at least one aqueous phase. Typically speaking, emulsions contain surfactants or surfactant-like materials which provide stability to the emulsions and inhibit de-phasing of the emulsions.

Surfactants

According to preferred embodiments, the compositions of the present invention further comprise at least one surfactant. Suitable surfactants for use in accordance with the present invention are not limited, and include any surfactant typically used in cosmetic compositions.

Examples of acceptable surfactants for use in the compositions of the present invention include, but are not limited to, nonionic surfactants such as linear or branched polyglycerol alkyl ethers, such as, for example, Polyglyceryl-2 oleyl ether or Polyglyceryl-4 oleyl ether; Ethoxylated alkylphenols having an alkyl chain comprising from 8 to 26 carbon atoms, for example ethoxylated alkylphenols having an alkyl chain comprising 9 carbon atoms (CTFA name: Nonoxynol), such as Nonoxynol-2, for example the product sold under the name Igepal CO-210 by Rhone-Poulenc, and their mixtures; Esters of polyols derived from fatty acids comprising from 8 to 30 carbon atoms, and their oxyalkylenated and in particular oxyethylenated derivatives, the polyols preferably being chosen from sugars, C2-C6 alkylene glycols, glycerol, polyglycerols, sorbitol, sorbitan, polyethylene glycols, polypropylene glycols and their mixtures.

Further specific examples include, glycerol esters, of monoglycerides, such as monoolein (glyceryl oleate); monolinolein (glyceryl linoleate); monolaurin (glyceryl laurate), and their mixtures; polyglycerol esters, of diglyceryl monoisostearate, diglyceryl oleate, triglyceryl monooleate, diglyceryl distearate, pentaglyceryl tristearate and their mixtures; oxyethylenated glycerol ester, of, for example, oxyethylenated glycerol stearate comprising 20 oxyethylene units, such as the product sold under the name Tagat S by Goldschmidt; sorbitan esters, of, for example, sorbitan stearate, such as the product sold under the name Span 60 by ICI, sorbitan laurate, such as the product sold under the name Span 20 by ICI, sorbitan palmitate, such as the product sold under the name Span 40 by ICI, sorbitan tristearate, such as the product sold under the name Span 65 by ICI, sorbitan oleate, such as the product sold under the name Span 80 by ICI, and sorbitan trioleate, such as the product sold under the name Span 85 by ICI. Mention may be made, as oxyethylenated sorbitan ester, of the Polysorbates and, for example, Polysorbate 21, sold under the name Tween 40 by ICI, and their mixtures; sugar esters, of those derived from the following sugars: sucrose, glucose, fructose, mannose, galactose, arabinose, xylose, maltose, cellobiose, lactose, trehalose, raffinose or gentianose (for example, of sucrose cocoate, sucrose monooctanoate, sucrose monodecanoate, sucrose monolaurate, sucrose monomyristate, sucrose monopalmitate, sucrose monostearate, sucrose monooleate, sucrose monolinoleate, sucrose dioleate, sucrose dipalmitate, sucrose distearate, sucrose dilaurate, sucrose dilinoleate, sucrose tristearate, esters of octyl-.beta.-glucofuranoside, the Galactolipids sold by Scotia Lipid Teknik, and their mixtures); and Polyol ethers derived from alcohols comprising from 8 to 30 carbon atoms and in particular sugar ethers, such as glucose ethers, for example, in particular, alkylpolyglucosides (APG), such as decylglucoside ((C9/C11 alkyl)polyglucoside (1,4)) such as the product sold under the name Mydol 10 by Kao Chemicals or under the name Plantaren 2000 UP by Henkel.

Suitable surfactants also include “alkoxylated fatty alcohols”—any fatty alcohol which comprises a carbon chain having at least 5 carbon atoms (excluding the alkoxy group) and at least one alkoxy group. For example, the at least one alkoxylated fatty alcohol may have a carbon chain of C8 or greater, C10 or greater, or C12 or greater. In one embodiment, the at least one alkoxylated fatty alcohol is chosen from ethoxylated fatty alcohols. In one embodiment, the at least one alkoxylated fatty alcohol is chosen from alkoxy esters of polyglyceryl.

Non-limiting examples of suitable alkoxylated fatty alcohols include ceteareth compounds (ceteareth-2, ceteareth-3, ceteareth-4, etc.), deceth compounds (deceth-3, deceth-5, deceth-6, etc.), laureth compounds (laureth-1, laureth-2, laureth-3, etc.), oleth compunds (for example, oleth-5), steareth compounds (for example, steareth-2, steareth-10), polyglyceryl 4-lauryl ether, polyglyceryl 4-oleyl ether, polyglyceryl 2-oleyl ether, polyglyceryl 2-cetyl ether, polyglyceryl 6-cetyl ether, polyglyceryl 6-oleylcetyl ether, polyglyceryl 6-octadecyl ether, C9-11 pareth-3, C9-11 pareth-6, C11-15 pareth-3, C11-15 pareth-5, and C11-15 pareth-12.

Suitable surfactants also include silicone surfactants such as alkoxylated or polyglycerolated, linear or branched, polydimethylsiloxanes. The alkoxylation may be within the polymer or present as a graft. Specific examples include, for example, polyglycerin modified silicones such as Polyglyceryl-3 Disiloxane Dimethicone, commercially available as KF-6100 from the Shin-Etsu Company; ethoxylated/propoxylated silicones such as PEG/PPG-18/18 Dimethicone, commercially available as DC 5225C from the Dow Corning Company, and Cetyl PEG/PPG-10/1 Dimethicone commercially available as ABIL EM90 from Goldschmidt; ethoxylated silicones such as PEG-10 Dimethicone commercially available as KF-6017 from the Shin-Etsu Company; and mixtures thereof.

Suitable surfactants also include natural surfactants such as phospholipids. As used in the present application, by the term phospholipid is meant a glycerol moiety on which two fatty acids are esterified at the carbon-1 and carbon-2 positions, and a phosphate group is esterified at the carbon-3 position. The fatty acid groups which are typically esterified to the glycerol may be saturated or unsaturated C12-C20 fatty acids, and are preferably selected from the group consisting of palmitic, stearic, oleic, linoleic, linolenic and arachidonic acids. The phosphate group may be substituted or unsubstituted. In its unsubstituted form, the phospholipid is phosphatidic acid. Substituents on the phosphate group are well-known to those of skill in the art and may include nitrogen-containing alcohols (e.g., ethanolamine, serine and choline) and inositol (hexahydroxycyclohexane). A preferred botanically-derived phospholipid is lecithin derived from soybeans. Lecithin may be hydrogenated, such that the unsaturated fatty acids in lecithin are converted to saturated fatty acids via hydrogenation.

If present, the at least surfactant is present in the compositions of the present invention in an amount ranging from about 0.5% to about 10% by weight, more preferably from about 1% to about 5% by weight, and most preferably from about 1.5% to about 4% by weight, based on the total weight of the composition, including all ranges and subranges within these ranges.

Coloring Agents

According to preferred embodiments of the present invention, compositions further comprising at least one coloring agent are provided. Preferably, such colored compositions can be cosmetic compositions such as, for example, lip compositions (for example, lipstick or liquid lip colors), mascaras, nail polish or foundations.

According to this embodiment, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%, including all ranges and subranges therebetween. In the case of certain products, the pigments, including nacreous pigments, may, for example, represent up to 50% by weight of the composition.

Additional Additives

The composition of the invention can also comprise any additive usually used in the field under consideration. For example, dispersants such as poly(l2-hydroxystearic acid), antioxidants, film forming agents, essential oils, sunscreens, preserving agents, fragrances, fillers, neutralizing agents, cosmetic and dermatological active agents such as, for example, emollients, moisturizers, vitamins, essential fatty acids, surfactants, silicone elastomers, pasty compounds, viscosity increasing agents such as waxes or liposoluble/lipodispersible polymers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

According to preferred embodiments of the present invention, compositions of the present invention are free of silicone oil: that is, they do not contain any silicone oil.

According to preferred embodiments of the present invention, methods of treating, caring for and/or making up keratinous material such as skin, lips, eyes and eyelashes by applying compositions of the present invention to the keratinous material in an amount sufficient to treat, care for and/or make up the keratinous material are provided. Preferably, “making up” the keratin material includes applying at least one coloring agent to the keratin material in an amount sufficient to provide color to the keratin material.

According to yet other preferred embodiments, methods of enhancing the appearance of keratinous material by applying compositions of the present invention to the keratinous material in an amount sufficient to enhance the appearance of the keratinous material are provided.

In accordance with the preceding preferred embodiments, the compositions of the present invention comprising at least one acrylic thickener and at least one high viscosity non-silicone oil are applied topically to the desired area of the keratin material in an amount sufficient to treat, care for and/or make up the keratinous material, to cover or hide defects associated with keratinous material, skin imperfections or discolorations, or to enhance the appearance of keratinous material. The compositions may be applied to the desired area as needed, preferably once or twice daily, more preferably once daily and then preferably allowed to dry before subjecting to contact such as with clothing or other objects (for example, a glass or a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to a preferred embodiment of the present invention, compositions having improved cosmetic properties such as, for example, improved feel upon application (for example, texture, reduced drag, spreadability, bounciness, creaminess and/or reduced tackiness), increased anti-smudging properties, increased pick up by the consumer during application (that is, amount of product “picked up” out of the container) and/or increased long wear properties are provided.

According to other embodiments of the present invention, methods of improving the anti-smudging, transfer-resistance, adherence and/or long wear properties of a composition, comprising adding at least one acrylic thickener and at least one high viscosity non-silicone oil to the composition are provided.

According to further embodiments of the present invention, methods of improving the feel or texture of a composition, preferably a makeup compositions such as a foundation or lip composition, comprising adding at least one acrylic thickener and at least one high viscosity non-silicone oil to the composition are provided

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

The following compositions were prepared.

| Ingredient | Example 1 | Example 2 |
|---|---|---|
| Viscup EZ | 6% | 1.5% |
| (sodium | | |
| acrylate/sodium | | |
| Acryloyldimethyl | | |
| Taurate | | |
| copolymer (and) | | |
| sorbitan laurate | | |
| (and) trideceth-6) | | |
| Diisostearyl | 12% | 12% |
| malate | | |
| (Schercemol | | |
| DISM) | | |
| PEG-10 | — | 0.75% |
| Dimethicone | | |
| Hydrogenated | — | 1.5% |
| Lecithin | | |
| Glyceryl Stearate | — | 1.5% |
| (and) PEG 100 | | |
| Stearate | | |
| Pigments (TiO₂ | 10% | 10% |
| and FeO₂) | | |
| Water | QS 100 | QS 100 |

The compositions were prepared as follows.

Ground pigments and Hydrogenated Lecithin in the water and the glycerin under the Silverson for 20 to 25 minutes.

Transferred the main batch to a mixer and added the remaining water phase ingredients (Preservatives and Humectants). Mixed while heating to about 65° C. to 70° C.

Combined all oil ingredients (Dimethicone, Caprylyl Methicone, PEG-10 Dimethicone, Glyceryly Stearate, and PEG 100 Stearate) in a side beaker and heated to about 70° C. while mixing under a Cowles blade until uniform and wax was melted.

Added Viscup EZ to the oil phase and mixed under a Cowles blade until uniform (about 10 minutes).

Transferred the main batch to a Greerco homogenizer. Added the oil to the water phase in the main beaker slowly while homogenizing at a moderately high speed. Continued homogenizing for 10 minutes until the batch was uniform.

Switched to the Rayneri chopper blade and mixed at a low speed while cooling the batch to 25° C.

This composition was also prepared by adding the Acrylic Thickener to the Oil phase instead of the water phase. The resulting product yielded a bouncier texture than by adding the acrylic thickener to the water phase.

### Example 3

The following composition was prepared.

| Ingredient | Example 3 |
|---|---|
| Viscup EZ | 6% |
| (sodium | |
| acrylate/sodium | |
| Acryloyldimethyl | |
| Taurate | |
| copolymer (and) | |
| sorbitan laurate | |
| (and) trideceth-6) | |
| Polybutene | 12% |
| (Indopol H 100) | |
| Pigments (TiO₂ | 10% |
| and FeO₂) | |
| Water | QS 100 |

What is claimed is:

1. A composition comprising at least one acrylic thickener and at least one high viscosity non-silicone oil, wherein the composition is free of silicone oil.

2. The composition of claim 1, wherein the at least one high viscosity non-silicone oil is a high viscosity ester oil.

3. The composition of claim 1, wherein the at least one high viscosity non-silicone oil is a high viscosity hydrocarbon oil.

4. The composition of claim 1, further comprising water.

5. The composition of claim 1, further comprising at least one coloring agent.

6. The composition of claim 4, wherein the composition is an emulsion.

7. The composition of claim 4, wherein the composition further comprises at least one surfactant.

8. The composition of claim 1, wherein the at least one acrylic thickener and the at least one high viscosity non-silicone oil are present in a weight ratio of from 0.01 to 1.

9. The composition of claim 1, wherein the at least one high viscosity non-silicone oil has a viscosity of at least 350 cSt at 25° C.

10. The composition of claim 2, wherein the at least one acrylic thickener and the at least one high viscosity ester oil are present in a weight ratio of from 0.01 to 1.

11. The composition of claim 2, wherein the at least one high viscosity ester oil has a viscosity of at least 350 cSt at 25° C.

12. The composition of claim 3, wherein the at least one acrylic thickener and the at least one high viscosity hydrocarbon oil are present in a weight ratio of from 0.01 to 1.

13. The composition of claim 3, wherein the at least one high viscosity silicone oil has a viscosity of at least 350 cSt at 25° C.

14. The composition of claim 1, wherein the acrylic thickener is sodium acrylate/sodium acryloyldimethyl taurate copolymer.

15. The composition of claim 1, wherein the acrylic thickener is present in an amount of 0.1% to 10% by weight of the weight of the composition.

16. A method of making up skin comprising applying the composition of claim 1 to the skin.

17. A method of making up lips comprising applying the composition of claim 1 to the lips.
